(19) United States
(12) Patent Application Publication
(10) Pub. No.: US 2017/0007754 A1
(43) Pub. Date: Jan. 12, 2017

(54) ENDOVASCULAR COMPLIANCE ASSEMBLY

(71) Applicant: Cook Medical Technologies LLC, Bloomington, IN (US)

(72) Inventors: Charles F. Babbs, West Lafayette, IN (US); Steven Charlebois, West Lafayette, IN (US); Kenneth Haselby, Battle Ground, IN (US); Jarin Kratzberg, Lafayette, IN (US); Justin Metcalf, West Lafayette, IN (US); Richard B. Sisken, West Lafayette, IN (US)

(21) Appl. No.: 14/791,712

(22) Filed: Jul. 6, 2015

(51) Int. Cl. A61M 1/10; A61F 2/844; A61M 1/12

(52) U.S. Cl. CPC A61M 1/1006; A61M 1/1046; A61M 1/125; A61F 2/844

(57) ABSTRACT

An endovascular assembly for improving vessel compliance by reducing the blood pressure needed to eject a given volume of blood. The assembly comprises a first expandable container, a balloon for example, positioned in the vascular system. The first container has a variable volume in response to blood flow in the vessel, and is fixed to at least one expandable attachment member. When the attachment member is expanded inside of the vasculature, the attachment member is preferably fixed inside the vessel. The assembly further comprises a second container, preferably having a fixed volume that forms a closed fluid system when fluidly connected to the first container. The connection between the first and second container permits a change in volume in the first container to flow fluid into the second container. The second container can be placed in a different location inside of the patient, preferably outside of the vessel.

### TECHNICAL FIELD

This invention relates generally to medical devices and particularly to an endovascular assembly for improving vascular compliance of a vessel.

### BACKGROUND OF THE INVENTION

When a vessel loses compliance, it loses elasticity and typically becomes stiffer. Vessels, such as the aorta, can lose compliance due to age, congestive heart failure, atherosclerosis, etc. As the aorta stiffens and loses compliance, the heart struggles to pump blood and must work harder to eject the same volume of blood from the left ventricle into the aorta with each heartbeat. If the heart is incapable of working harder because of underlying diseases, then less blood will be ejected into the aorta with each heartbeat.

### SUMMARY OF THE INVENTION

In one preferred and illustrative embodiment, an endovascular assembly is positionable in the descending aorta of a patient advantageously to improve the compliance of the vessel. As a result, the blood pressure required to pump blood through the vascular system is lowered and the work the heart performs to pump the same volume of blood through the vascular system is reduced. If the work of the heart remains constant, then a greater volume of blood will be pumped.

To accomplish this, the endovascular assembly in the preferred and illustrative embodiment includes a first container, such as an expandable balloon, that is positionable within a vessel such as, for example, the descending aorta. The first container is fixedly positionable within the vessel with at least one attachment member. The first container has a shape and a volume at least one thereof that is variable and changes as blood in the vessel flows thereby. The first container is fluidly connectable to a second container, which is preferably located outside of the vessel, to form a closed fluid system. The connecting fluid may be a gas such as air or carbon dioxide.

When the first container and second container are connected and inflated with a volume of fluid such as a gas or a liquid, the second container acts as a reservoir for fluid to flow in and out of the first container. The first container decreases in volume as blood is expelled from the left ventricle of the heart and flows over the first container. When the balloon decreases in volume, fluid in the first container flows into the second container. In a preferred embodiment of the present invention, the second container has a fixed volume, and a rigid wall. In an alternative embodiment of the present invention the second container has an elastic wall and a variable volume.

Advantageously, the second container is implantable and preferably placed in the abdomen or subcutaneous tissue of the leg, requiring no external bodily connections to a power source or pump. By not relying on external connections, the endovascular assembly functions as a passive pump and preferably used for more long-term care, particularly in ambulatory patients. Moreover, the second container is preferably divided into two compartments, an inner inflatable chamber that is directly connected to the first container, and an outer chamber. The outer chamber preferably includes a port which advantageously allows for a physician to make adjustments, such as fluid volume, to the endovascular assembly after initial implantation. In this embodiment, the fluid in the inner chamber and first container would preferably be carbon dioxide or another gas safe for use in the bloodstream, while the fluid in the outer chamber could be the same or another fluid.

The attachment member is preferably a self-expanding stent located externally to the first container. Preferably, there are two self-expanding stents connected to the proximal and the distal ends of the first container. In another embodiment, the attachment member is preferably a self-expanding balloon, located internally in and/or integrally with the first container.

The balloon preferably has an outer shape with at least two planar surfaces. Having at least two planar surfaces has the advantage of lessening the probability of blood clots dislodging from the surface of the balloon.

### BRIEF DESCRIPTION OF THE DRAWING

The invention may be more fully understood by reading the following description in conjunction with the drawings, in which:

FIG. 1 depicts a side plan view of an illustrative embodiment of an endovascular assembly within the descending aorta of a patient, depicting a first container within the aorta connected to a second container located in the abdomen.

FIG. 2 depicts a side plan view of an intra-aortic balloon and attachment members of the endovascular assembly of FIG. 1.

FIGS. 3A-3C depict cross-sectional views of the intra-aortic balloon of FIG. 2 within a vessel.

FIG. 4 depicts an enlarged side plan view of the endovascular assembly of FIG. 1 including the intra-aortic balloon and an extravascular container.

FIGS. 5A-5B depict side plan views of the endovascular assembly including the intra-aortic balloon and the extravascular container with an outer chamber and an inner inflatable chamber inflated with a fluid such as a gas or a liquid.

FIGS. 6A-6C depict a side plan view of the endovascular assembly with an external attachment member(s), and two cross sectional views of the intra-aortic balloon with alternative stent placements.

### DETAILED DESCRIPTION

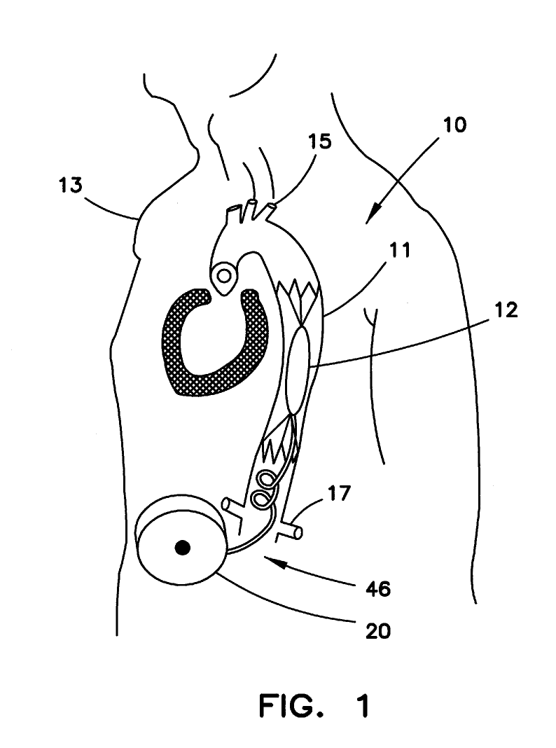

Now looking at the drawings and in particular FIG. 1, an illustrative embodiment of an endovascular assembly 10 is depicted positioned in the descending aorta 11 of a patient 13. The endovascular assembly 10 includes a first container 12 that is expandable and is placed in the aorta. A second container 20 located outside of the vasculature is placed subdermally and connected to the first container 12 to form a closed fluid system 46. The first and second containers are inflated with a volume of fluid such as a gas or a liquid. The second container 20 serves as a reservoir for fluid to flow into and out of the first container 12. As the first container 12 decreases in volume and collapses under the increasing pressure of blood being ejected from the left ventricle, a volume of fluid in the first container 12 flows into the second container 20. By collapsing under increasing pressure of the passing blood, the first container 12 adds compliance to the aorta which allows the heart to eject the same amount of blood at a reduced blood pressure or a greater volume of blood at a constant pressure. The movement of fluid between the first container 12 and the second container 20 is ideally passive as according to Boyle's law, P₁V₁=P₂V₂ (P=pressure and V=volume) In a closed system a change in fluid pressure or volume in one chamber will result in the equivalent change in fluid pressure or volume in the connected chamber. Additionally the fluid volume in the first container 12 will change with changes in pressure at a given temperature to maintain equilibrium, according to the ideal gas law PV=nRT (P=pressure, V=volume, n=number of moles of fluid, R=ideal gas constant, T=temperature).

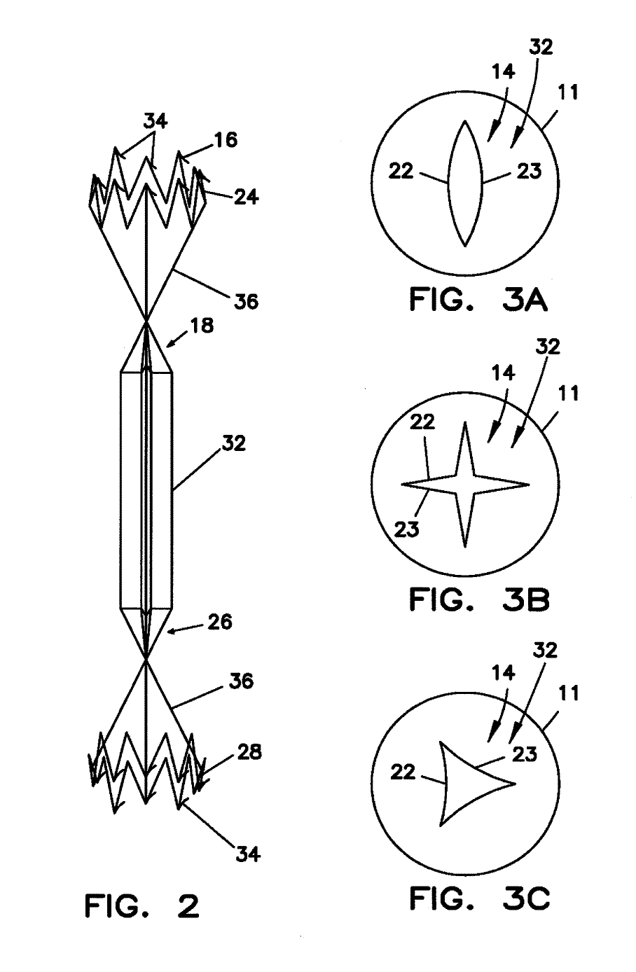

FIG. 2 depicts a side plan view of the intra-aortic balloon of endovascular assembly 10 of FIG. 1. The intra-aortic balloon 32 has a proximal end 18 and distal end 26 and a diameter and cross sectional shape that allows blood to flow past it in the aorta 11.

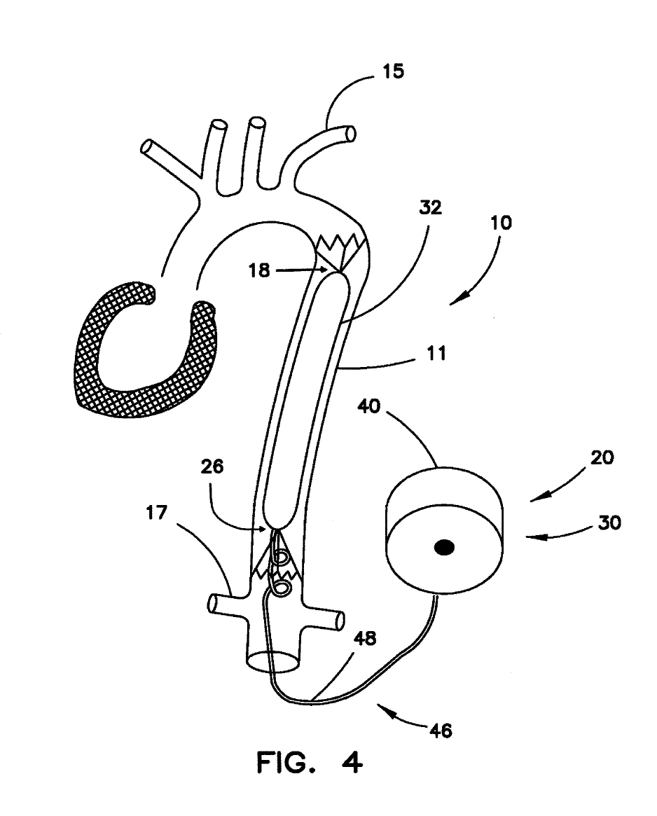

FIGS. 3A-C depict the balloon 32 of FIG. 2 having an outer shape 14 with at least two planar surfaces 22 and 23. It is also desirable to have a balloon with multiple planar surfaces 22 and 23, such as a star shaped balloon 32, depicted in FIGS. 2 and 3B, or a triangular shaped balloon 32, depicted in FIG. 3C. It is advantageous to have a balloon shaped with at least two generally planar surfaces 22 and 23, although some curvature is permitted. Balloons with generally planar surfaces, as opposed to cylindrical or spherical balloons, are preferable as they commonly prevent clots from dislodging from the surface of the balloon. Additionally, it is desirable for both the proximal and distal portions of the balloon to be tapered to lessen resistance to blood flow.

The intra-aortic balloon 32 is preferably made of polymer materials such as a polyamide-polyimide blend, Thoralon®, silicone, or nylon; however, it should be understood that a variety of other commercially available materials can be used.

Referring now to FIG. 2, it can be seen that the intra-aortic balloon 32 is connected to at least one attachment member 16. The attachment member is preferably a self-expanding stent 24. The stent 24 serves to anchor and center the balloon 32 within the aorta. The stent 24 can preferably have a Z-stent configuration or cannula cut stent (U.S. Pat. No. 7,905,915 and U.S. Pat. No. 7,172,623). The stent 24 is preferably made of nitinol, but can be made out of stainless steel, or any other suitable commercially available material for endovascular stents. Further, it may be advantageous for the stent 24 to contain barbs 34 that engage with the wall of the aorta to enhance attachment to the aortic wall.

There is at least one self-expanding stent 24 connected to the proximal end 18 of the intra-aortic balloon 32, or preferably two self-expanding stents with the second stent 28 connected to the distal end 26 of the balloon 32. The balloon 32 is connected to the self-expanding stents by tethers 36. The tethers 36 may be non-resorbable, commercially available sutures of any suitable size made out of a variety of biocompatible materials. However, it may be advantageous to use chromic sutures that can be broken with a balloon. This would allow for the endovascular assembly 10 to be removed if it should become necessary sometime after implantation.

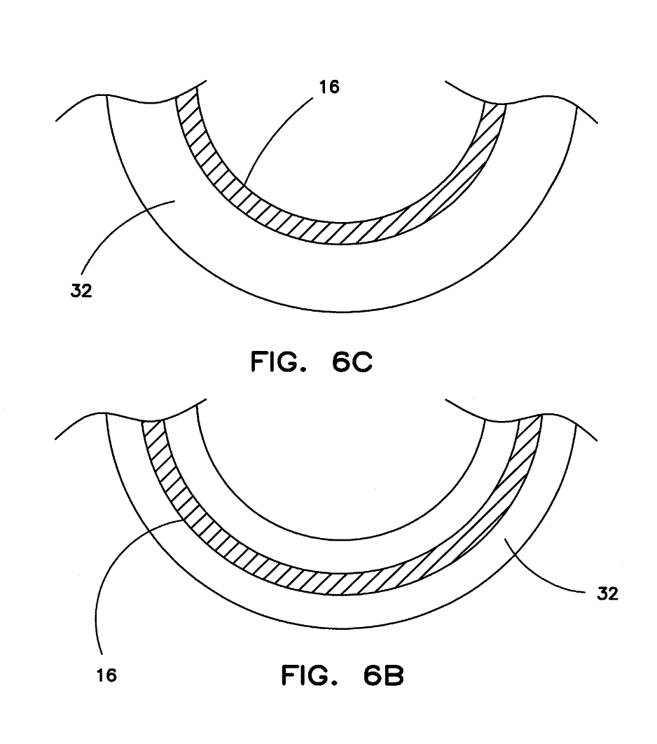

Alternatively, in another aspect of the present invention, depicted in FIG. 6A-C, the connectable attachment member 16 may be integrated into the design of the intra-aortic balloon 32 itself. Where connectable, refers to circumferentially, longitudinally, internally, or externally connectable to the intra-intra-aortic balloon 32. The attachment member 16 is a self-expanding stent located internally to FIG. 6C or as in FIG. 6B in the wall of the intra-aortic balloon 32. An integrated stent and balloon construct would not require tethers and would allow the balloon material to flex as needed while the integrated stent maintains a grip on the wall of the aorta. The stent is preferably self-expanding and can be flat or rounded where it contacts the wall of the aorta. The integrated stent is preferably made of nitinol, or any other suitable commercially available stent material, to allow the integrated stent and balloon construct to be folded like a conventional balloon for delivery. In another aspect of the present invention, the endovascular assembly 10 comprises no attachment members 16.

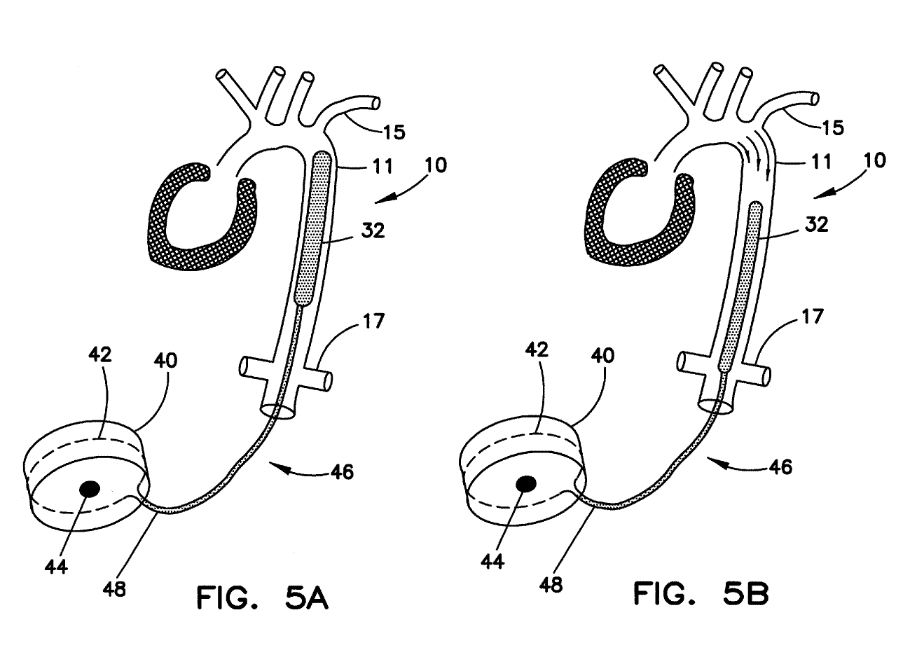

FIG. 4 depicts an enlarged side plan view of the endovascular assembly 10 of FIG. 1 including the intra-aortic balloon 32 and an extravascular container 30. The intra-aortic balloon 32 is endovascularly positioned in the descending aorta 11 and is placed so that its proximal end 18 lies just distal to the subclavian arteries 15, and its distal end 26 lies just above the renal arteries 17. The length of the balloon 32 for a typical adult would be approximately 20 centimeters in length, but may be adjusted to suit different patient vascular anatomies. Positioned in such a manner, clots that may form and dislodge from the balloon would avoid the brain. Furthermore, concerns regarding blockage of renal or mesenteric arteries are reduced.

It can be seen that the intra-aortic balloon 32, as depicted in FIG. 4, is fluidly connected to the second container 20, or extravascular container 30, by a hollow flexible tube 48. The connection between the intra-aortic balloon 32 and extravascular container 30 forms a closed fluid system 46 such that fluid, such as a gas or a liquid, within the balloon 32 can flow into the extravascular container 30, or fluid within the extravascular container 30 can flow into the intra-aortic balloon 32, but the fluid will not enter the vascular system. The diameter of the tube or catheter 48 connecting the extravascular container 30 will typically be at least 2 mm to avoid introducing significant resistance to fluid flow between the balloon 32 and the extravascular container 30. The catheter 48 may also have a curved or spiraled portion to avoid placing the aorta and iliac arteries under undue longitudinal stress.

The intra-aortic balloon 32 is inflated with a fluid such as a gas or a liquid. The balloon 32 is preferably inflated with carbon dioxide or any other suitable fluid that is impermeable to the material of the balloon. Carbon dioxide is preferred for safety, as it would rapidly dissolve into the bloodstream in the event of system rupture.

The extravascular container 30 is preferably implanted subdermally, preferably below the surface of the abdomen. The catheter 48 can be tunneled from the location that it exits the aorta to the extravascular container 30. In another embodiment of the present invention, the extravascular container 30 can be implanted in the patient's 13 shoulder or chest area, allowing the catheter 48 to travel through the subclavian artery and the balloon 32 to hang freely generally below the container 30, such as in the descending aorta 11. The extravascular container 30 and or outer chamber 40 can be of either elastic or rigid material in this embodiment. Where the extravascular container 30 and or chamber 40 are elastic, their volumes will be variable.

The outer chamber 40 of the extravascular container 30 is preferably made out of an elastic or rigid material that will not burst or rupture upon a forceful impact that could result, for example, by a patient falling. Thus, the outer chamber 40 can be made out of polyether ether ketone (PEEK), high density polyethylene (HDPE), a polyamide-polyimide blend, or any other similar commercially available material suitable for implantation within a human patient.

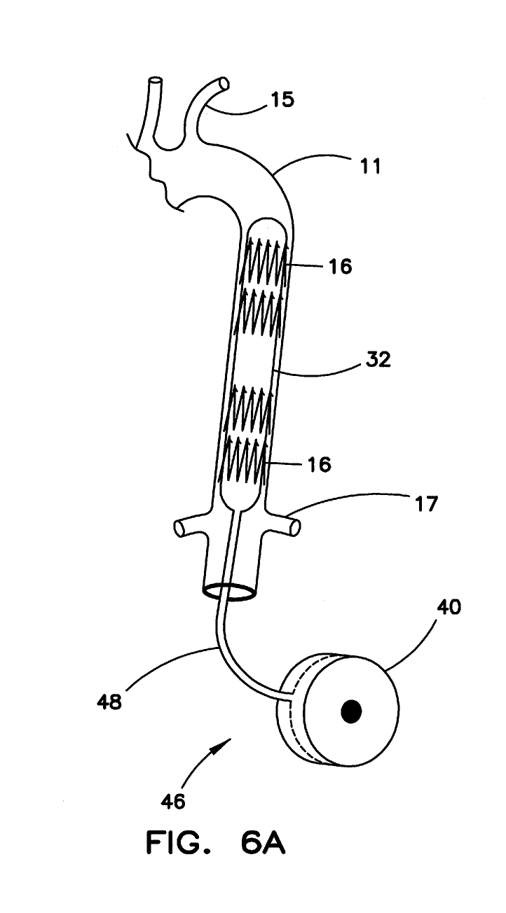

FIGS. 5A-5B depict side plan views of the endovascular assembly of the present invention including the intra-aortic balloon and the extravascular container with an outer chamber and inner inflatable chamber inflated with a gas or a liquid. It can be seen that the extravascular container 30 is preferably divided into two compartments. The first compartment, or inner inflatable chamber 42, is directly connected to the intra-aortic balloon 32 and resides inside the larger second compartment, or outer chamber 40. The inner inflatable chamber 42 can be made of the same material as the intra-aortic balloon 32. The inner inflatable chamber 42 isolates the fluid in the outer chamber from the fluid in the catheter 48 and intra-aortic balloon 32. This acts as a safety feature in the event of a balloon rupture. It also allows for different fluids to be utilized in the balloon 32 and inner inflatable chamber 42 than in the outer chamber 40. For example, the balloon 32 and inner inflatable chamber 42 can be inflated with carbon dioxide while the outer chamber 40 can be inflated with another fluid. This may be advantageous as it may be convenient for adjusting the pressure of the endovascular assembly to meet patient needs.

In this regard, it would be desirable to include a port 44 on the outer chamber of the second container that is accessible external to the patient. This would be advantageous to allow for adjustment of the pressure in the closed fluid system 46. The port can be a septum or an infusion port 44 similar to the Vital-Port Titanium Power-Injectable Vascular Access System manufactured by COOK Medical Technologies, Bloomington, Ind. As the outer chamber is implanted subdermally in the abdomen or thigh, a physician can easily access the port in order to increase or decrease the pressure of gas or liquid in the closed fluid system. This could be done in an outpatient procedure.

Another possible feature of the extravascular container 30 involves one or more pressure sensors (not shown) and sufficient electronics to transmit the pressures measured through the patient to an external reader. The electronics may be self-powered with batteries or energy harvesting technologies (e.g. piezoelectric or photovoltaic systems) or powered by radio frequency or other electromagnetic energy from the external reader or other external source.

When the intra-aortic balloon 32 and the inner inflatable chamber 42 of the extravascular container 30 are connected as depicted in FIGS. 5A and 5B, a closed fluid system 46 is formed. The balloon 32 is inflated with a volume of gas or liquid in the range of about 50 mL to about 130 mL. The outer chamber 40 would have a volume capacity in the range of about 80 mL to about 120 mL and the inner inflatable chamber would have a volume in the range of about 60 mL and about 95 mL, or approximately 80 to 90% of the total capacity of the outer chamber. Initially, the intra-aortic balloon (32) and inner inflatable chamber 42 can be inflated with carbon dioxide. Sometime after deployment, it may desirable to adjust the volume of fluid in the endovascular assembly 10. To achieve this, the outer chamber 40 can be inflated with for example, air, or any other suitable fluid, via the infusion port 44 provided on the outer chamber 40. It may be advantageous to inflate the outer chamber 40 with air as this could be done in a simple outpatient procedure.

When the intra-aortic balloon 32 and the inner inflatable chamber 42 of the extravascular container are connected and inflated in the above described manner and as depicted in FIG. 5A, the balloon 32 acts as passive pump requiring no open wounds to connect the balloon to an external pump or power supply. The extravascular container 30 serves as a volume compensating reservoir for the intra-aortic balloon 32. As the balloon collapses, depicted in FIG. 5B under the increasing pressure of blood being ejected from the heart, a volume of fluid in the balloon 32 flows into the inner inflatable chamber 42 of the extravascular container 30. By collapsing under increasing pressure, the balloon 32 adds compliance to the aorta which allows the heart to eject the same amount of blood with less pressure or to eject a greater amount of blood with the same pressure.

While preferred embodiments of the invention have been described, it should be understood that the invention is not so limited, and modifications may be made without departing from the invention.

What is claimed is:

1. An endovascular assembly for improving vascular compliance comprising:
a first container being expandable and endovascular, when expanded in a vessel, the first container has a volume changing in response to blood flowing thereby;
a second container being implantable and fluidly connectable to the first container; and
wherein when connected, the first container and the second container form a closed fluid system, whereby a change in volume of the first container due to blood flowing thereby is accompanied by a change in pressure of the closed fluid system.

2. The endovascular assembly of claim 1 further comprising at least one self-expanding stent connectable to the first container, when expanded in a vessel, the attachment member is fixedly positioned therein.

3. The endovascular assembly of claim 2 wherein the second container has a fixed volume.

4. The endovascular assembly of claim 1 wherein the second container has a variable volume.

5. The endovascular assembly of claim 2 wherein the first container comprises an expandable balloon.

6. The endovascular assembly of claim 2 wherein the first container has an outer shape with at least two planar surfaces.

7. The endovascular assembly of claim 2 wherein the at least one attachment member is a self-expanding stent.

8. The endovascular assembly of claim 2 wherein the second container comprises an outer chamber and an inner inflatable chamber contained within the outer chamber, and wherein the inner chamber is fluidly connectable to the distal end of the first container.

9. The endovascular assembly of claim 8 wherein the outer chamber of the second container comprises a port accessible external to a patient.

10. The endovascular assembly of claim 2 further comprising a second attachment member connectable to the first container.

11. The endovascular assembly of claim 2 wherein the attachment member is integrated in a wall of the first container.

12. An endovascular assembly for improving vascular compliance comprising:
a first container being expandable and endovascular, when expanded in a vessel, the first container has a volume changing in response to blood flowing thereby;
the first container having an outer shape with at least two planar surfaces;
at least one self-expanding stent connectable to the first container, when expanded in a vessel, the attachment member is fixedly positioned therein;
a second container being implantable and fluidly connectable to the first container; and
wherein when connected, the first container and the second container form a closed fluid system, whereby a change in volume of the first container due to blood flowing thereby is accompanied by a change in pressure of the closed fluid system.

13. The endovascular assembly of claim 12 wherein the second container has a fixed volume.

14. The endovascular assembly of claim 12 wherein the second container has a variable volume.

15. The endovascular assembly of claim 13 wherein the first container comprises an expandable balloon.

16. The endovascular assembly of claim 15 wherein the second container comprises an outer chamber and an inner inflatable chamber contained within the outer chamber, and wherein the inner chamber is fluidly connectable to the distal end of the first container.

17. The endovascular assembly of claim 16 wherein the outer chamber of the second container comprises a port accessible external to a patient.

18. The endovascular assembly of claim 17 further comprising a second self-expanding stent connectable to the first container.

19. The endovascular assembly of claim 12 wherein the attachment member is integrated into at least one planar surface of the attachment member.

20. An endovascular assembly for improving vascular compliance comprising:
an expandable balloon, the balloon being endovascular, when expanded in a vessel, the balloon has a variable volume changing in response to blood flowing thereby;
the balloon having an outer shape with at least two planar surfaces;
a first self-expanding stent connectable to the expandable balloon and a second self-expanding stent connectable to the expandable balloon, when the stents are expanded in a vessel, the stents are fixedly positioned therein;
a container comprising an outer chamber and an inner inflatable chamber contained within the outer chamber, the inner chamber is fluidly connectable to the expandable balloon, and
wherein when connected, the expandable balloon and the inner chamber form a closed fluid system, whereby a change in volume of the balloon due to blood flowing thereby is accompanied by a change in pressure of the closed fluid system.
